(19) United States
(12) Patent Application Publication
(10) Pub. No.: US 2006/0200032 A1
(43) Pub. Date: Sep. 7, 2006

(54) Linear oscillation pressurization type electronic sphygmomanometer

(75) Inventor: Tower S.J. Wu, Taipei (TW)

Correspondence Address: HDSL, 4331 STEVENS BATTLE LANE, FAIRFAX, VA, 22033, US

(73) Assignee: Rossmax International Ltd.

(21) Appl. No.: 11/367,263

(22) Filed: Mar. 3, 2006

(30) Foreign Application Priority Data
Mar. 4, 2005 (TW) 094106588

(51) Int. Cl. A61B 5/02

(52) U.S. Cl. 600/493

(57) ABSTRACT

A linear oscillation pressurization type electronic sphygmomanometer includes a main body enclosing a CPU; a pressurization unit arranged in the main body and controlled by the CPU and providing a linear oscillation pressurization operation, a cuff connected to the linear oscillation pressurization unit; a pressure controller controlling pressure variation in the cuff to facilitate data collection of sensor, a sensor connected to the cuff and can be at least one of pressure sensor or Korotkff's sound sensor, or both; a display arranged on the main body and showing the blood pressure measurement result. The CPU provides a digital signal to control a reciprocating operation of the pressurization unit to facilitate the blood pressure measurement of the cuff.

### BACKGROUND OF THE INVENTION

1. Field of the Invention

The present invention relates to an electronic sphygmomanometer, especially to a linear oscillation pressurization type electronic sphygmomanometer using reciprocating operation.

2. Description of Prior Art

FIGS. 1 and 2 show the prior art of electronic sphygmomanometer and the pressurization unit thereof. The electronic sphygmomanometer comprises central processing unit (CPU) 9, a rotary type pressurization unit 91, a pressure controller 92, a cuff 93, a pressure sensor 94 and a display 95, which are arranged in a main body of the electronic sphygmomanometer. For the operation of the pressurization unit, the CPU 9 controls the rotary type pressurization unit 91 to send pressured air to the cuff 93. The rotary type pressurization unit 91 comprises a micro rotary motor and a pump driven by the micro rotary motor. The motor is driven by an ON/OFF signal and the waveform of the ON/OFF signal is shown in FIG. 2. The pressurizing process is controlled by controlling pressurization time of the motor and pump, thus the flow rate of the pumping air cannot be controlled. The motor will keep running and by the shifts; it will change the piston/membrane direction to keep the piston moving up and down, then the pressure difference caused by the piston will make the inlet/outlet valves open and close to finish a pumping cycle. However, the above-mentioned pressurization unit for the electronic sphygmomanometer uses shifts for force transfer. The power output by the motor will be wasted on the force transfer of the shifts. Moreover, the shifts used for pump are bulky for device demanding compact size such as sphygmomanometer, especially for sphygmomanometer used for arm or finger. Because of the inertia of the rotor inside the rotary motor, the control of the pressurization rate is difficult, especially when the pressurization rate is instantly reduced from the high speed to the low speed.

Moreover, the conventional rotary motor uses electrical brush to guide electrical current to the rotor. The noise and electromagnetic radiation will be generated due to the friction between electrical brush and rotor, they are not good for the medical instrument application.

### SUMMARY OF THE INVENTION

The present invention is to provide a linear oscillation pressurization type electronic sphygmomanometer. The pressurization of the electronic sphygmomanometer is achieved by a linear oscillation pressurization unit. The linear oscillation pressurization unit could be directly controlled by a serial of digital oscillation current pulse signals that from the CPU; or a control circuit that combines the electronic sphygmomanometer's circuit and an external circuit. Moreover, the control circuit of the oscillation pressurization unit 2 could be an external circuit that independent from the electronic sphygmomanometer's circuit. A reciprocating pressurization is exerted on the cuff, this will make it easier to control the pumping rate and more comfortable during the measurement period. Moreover, because the brush was removed, there is less noise and electrical radiation in this design. Therefore, the electronic sphygmomanometer according to the present invention has low cost and small size for using in wrist-type and finger-type electronic sphygmomanometer.

The linear oscillation pressurization type electronic sphygmomanometer according to the present invention comprises:

a main body;

a CPU arranged in the main body

a CPU controlling the operation of the electronic sphygmomanometer and outputting serial digital oscillation current pulses;

a pressurization unit arranged in the main body and driven by the serial digital oscillation current pulses output from the CPU; or by a control circuit; where the control circuit is arranged inside the pressurization unit or an independent circuit outside the pressurization unit , thus generating a pressurized air to the cuff;

a cuff connected to the pressurization unit

a pressure controller driven by the CPU to control pressure variation of cuff during the measurement of blood pressure;

a sensor connected to the cuff, where the sensor comprises at least one of pressure sensor or Korotkff's sound sensor, or both

a display arranged on a main body to display the blood measurement result.

### BRIEF DESCRIPTION OF DRAWING

The features of the invention believed to be novel are set forth with particularity in the appended claims. The invention itself however may be best understood by reference to the following detailed description of the invention, which describes certain exemplary embodiments of the invention, taken in conjunction with the accompanying drawings in which:

FIG. 1 shows the prior art electronic sphygmomanometer and the pressurization unit thereof.

FIG. 2 shows the signal waveform of the pressurization unit of prior art electronic sphygmomanometer, t1 is pressurization time of rotary motor/pump, t2 and t3 are pressurization period of linear oscillation pressurization unit, where t1>>t2, t3.

FIG. 3 shows a schematic diagram of the linear oscillation pressurization type electronic sphygmomanometer of the present invention.

FIG. 4 shows the signal waveform of the pressurization unit of electronic sphygmomanometer of the present invention, where t2 is pressurization period of linear oscillation pressurization unit.

FIG. 5 shows another signal waveform of the pressurization unit of electronic sphygmomanometer of the present invention, where t3 is pressurization period of linear oscillation pressurization unit.

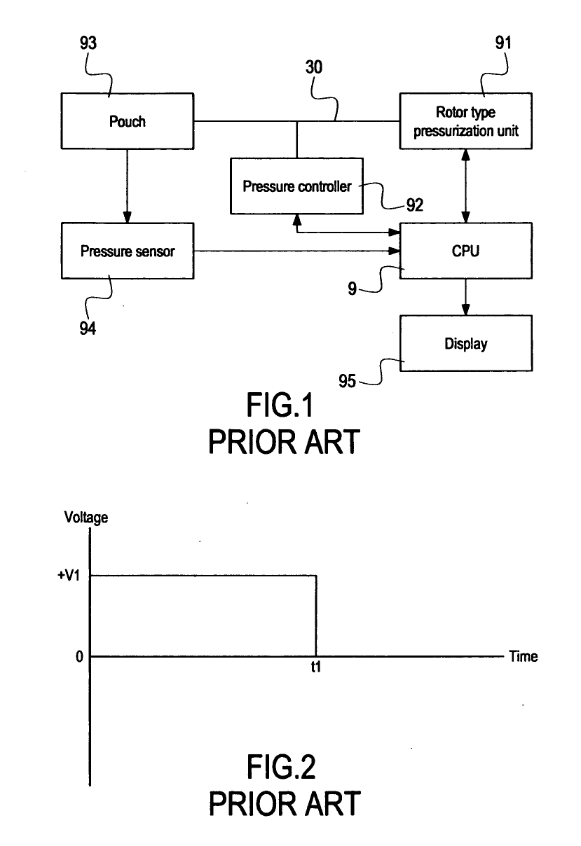

### DETAILED DESCRIPTION OF THE INVENTION

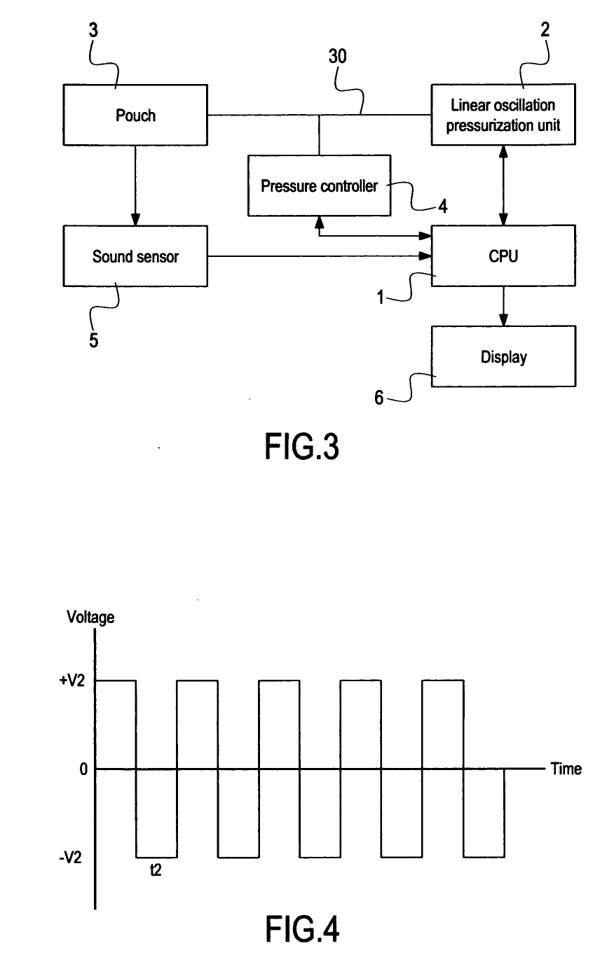

With reference to FIGS. 3 to 5, a linear oscillation pressurization type electronic sphygmomanometer is disclosed according a preferred embodiment of the present invention. The electronic sphygmomanometer comprises a CPU 1, a linear oscillation pressurization unit 2 connected to the CPU 1, a cuff 3 connected to the linear oscillation pressurization unit 2, a pressure controller 4 connected between the linear oscillation pressurization unit 2 and the cuff 3, a pressure/Korotkff's sound sensor 5 and a display 6, which are arranged in a main body of the electronic sphygmomanometer, as shown in FIG. 3.

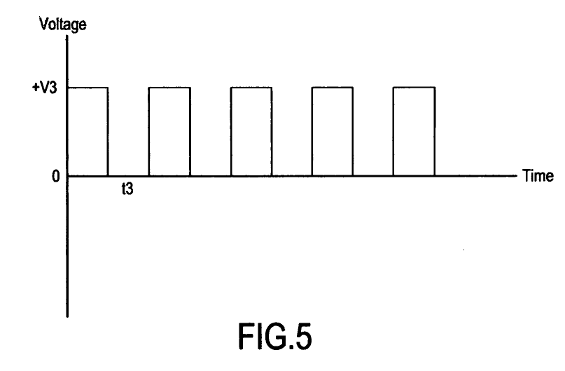

The CPU 1 arranged in the main body of the electronic sphygmomanometer is used to control measurement of the whole electronic sphygmomanometer and generate serial oscillation signal to drive the linear oscillation pressurization unit 2. The CPU 1 also drives the pressure controller 4 to control the pressure variation of the cuff 3 in the operation of the electronic sphygmomanometer to collect data for the pressure/Korotkff's sound sensor 5 and displays the measurement result. The CPU 1 is one of CPU or MPU, and generates periodic serial oscillation current signals upon control. The periodic serial oscillation current signals can be oscillation current signals composed of positive pulse and negative pulse as shown in FIG. 4 or continuous positive pulses or negative pulses (FIG. 5 shows the example of positive pulses) to control the pressurization of the linear oscillation pressurization unit 2.

The linear oscillation pressurization unit 2 comprises a permanent magnetic element and an electromagnetic element to generate reciprocating magnetization action. Therefore, pressurized air is supplied to the cuff 3. FIG. 4 shows the wave of operation signal for the linear oscillation pressurization unit 2, which are oscillation current pulses composed of continuous positive pulses and negative pulses.

According to another preferred embodiment of the present invention, the linear oscillation pressurization unit 2 comprises a permanent magnetic element, an electromagnetic element and an elastic element to generate reciprocating magnetization action. Similarly, the linear oscillation pressurization unit 2 receives serial digital oscillation current pulses to drive the electromagnetic element and the elastic element to have reciprocating movement and generate pressured air to the cuff 3. FIG. 5 shows the waveform of operation signal for the linear oscillation pressurization unit 2, which are oscillation current pulses composed of continuous positive pulses. It should be noted that the operation signal for the linear oscillation pressurization unit 2 could also be composed of continuous negative pulses.

The driving of the linear oscillation pressurization unit 2 can be controlled by current pulses output from the CPU, where the linear oscillation pressurization unit 2 does not have control circuit. Alternatively, the linear oscillation pressurization unit 2 could be controlled by a control circuit which combines the electronic sphygmomanometer's circuit and an external circuit. Moreover, the control circuit of the oscillation pressurization unit 2 could be an external circuit that independent from the electronic sphygmomanometer's circuit.

The cuff 3 is a pressurizable cuff, which can be clamped to user and is connected to a sensor. The inlet of the cuff 3 is connected to the outlet of the pressurization unit 2 through a gas tube 30 to obtain air for the blood pressure measurement.

The pressure controller 4 is connected between the pressurization unit 2 and the gas tube 30 of the cuff 3. As shown in FIG. 3, the pressure controller 4 is electrically connected to the CPU 1 to control pressure variation of the cuff 3 during the measurement.

The pressure/Korotkff's sound sensor 5 is connected to the cuff 3 and the CPU 1 to sense the blood pressure when the cuff 3 is degassed. The pressure/Korotkff's sound sensor 5 comprises a pressure sensor, a Korotkffs sound sensor, or both of them.

The display 6 is arranged on one face of the main body and connected to the CPU to display the blood pressure measurement result in the cuff 3.

In the above-mentioned electronic sphygmomanometer, the pressurization unit 2 is driven by a serial digital oscillation current pulses from the CPU 1 to provide reciprocating operation to the cuff 3 for the blood pressure measurement. The electronic sphygmomanometer according to the present invention does not need rotary motor in comparison with prior art electronic sphygmomanometer. Therefore, the electronic sphygmomanometer according to the present invention has low cost and small size for using on wrist-type and finger-type electronic sphygmomanometer.

Although the present invention has been described with reference to the preferred embodiment thereof, it will be understood that the invention is not limited to the details thereof. Various substitutions and modifications have suggested in the foregoing description, and other will occur to those of ordinary skill in the art. Therefore, all such substitutions and modifications are intended to be embraced within the scope of the invention as defined in the appended claims.

What is claimed is:

1. A linear oscillation pressurization type electronic sphygmomanometer, comprising
a CPU;
a pressurization unit connected to the CPU;
a cuff connected to the linear oscillation pressurization unit;
a pressure controller connected between the linear oscillation pressurization unit and the cuff;
a sensor between the cuff and the CPU; and
a display arranged on a main body to display blood measurement results.

2. The linear oscillation pressurization type electronic sphygmomanometer as in claim 1, wherein the sensor is one of pressure sensor and Korotkff's sound sensor.

3. The linear oscillation pressurization type electronic sphygmomanometer as in claim 1, wherein the sensor comprises pressure sensor and Korotkff's sound sensor.

4. The linear oscillation pressurization type electronic sphygmomanometer as in claim 1, wherein the pressurization unit is a linear oscillation pressurization unit.

5. The linear oscillation pressurization type electronic sphygmomanometer as in claim 1, wherein the pressurization unit is controlled by a serial digital oscillation current pulses.

6. The linear oscillation pressurization type electronic sphygmomanometer as in claim 5, wherein the serial digital oscillation current pulses comprises continuous positive pulses and negative pulses.

7. The linear oscillation pressurization type electronic sphygmomanometer as in claim 5, wherein the serial digital oscillation current pulses comprises continuous positive pulses.

8. The linear oscillation pressurization type electronic sphygmomanometer as in claim 5, wherein the serial digital oscillation current pulses comprises continuous negative pulses.

9. The linear oscillation pressurization type electronic sphygmomanometer as in claim 1, wherein the pressurization unit further comprises a controller circuit and working with a controller of the electronic sphygmomanometer to control the pressurization unit.

10. The linear oscillation pressurization type electronic sphygmomanometer as in claim 1, wherein a control circuit is arranged outside the electronic sphygmomanometer's circuit to control the pressurization unit.

11. The linear oscillation pressurization type electronic sphygmomanometer as in claim 1, wherein the CPU is connected to a display.

12. A linear oscillation pressurization type electronic sphygmomanometer, comprising a pressurization control unit, the pressurization control unit comprising:
a CPU;
a pressurization unit connected to the CPU;
a cuff connected to the linear oscillation pressurization unit; and
a pressure controller connected between the linear oscillation pressurization unit and the cuff.
